(19) United States
(12) Patent Application Publication
(10) Pub. No.: US 2017/0089886 A1
(43) Pub. Date: Mar. 30, 2017

(54) IN VITRO DIAGNOSTIC FOR MODEL-BASED THERAPY PLANNING

(71) Applicant: CYTOCENTRICS, INC., U.S.A., San Antonio, TX (US)

(72) Inventors: Thomas KNOTT, Rostock (DE); Andrea WELLING, Munchen (DE)

(21) Appl. No.: 15/312,524

(22) PCT Filed: May 21, 2015
(86) PCT No.: PCT/EP2015/061317

§ 371 Date: Nov. 18, 2016

(30) Foreign Application Priority Data
May 21, 2014 (DE) 102014209627.8

(51) Int. Cl. G01N 33/50; G01N 33/487

(52) U.S. Cl. CPC G01N 33/5061; G01N 33/48728; G01N 2800/326; G01N 2800/325; G01N 2800/52

(57) ABSTRACT

A compact in vitro diagnostic (WD) device is used to provide in vitro model-based diagnosis and a basis for model-based therapy planning. The IVD device and an associated procedure may be used to assess the effect of drugs on the electro-physiological phenotype of cardiomyocytes or any cell of a patient. The effects may be assessed in a clinical environment. The assessed information for the patient may be combined with clinical data for the patient and provided to a database. The database may collect the assessment results and clinical patient data for a plurality of patients. The information in the database may be used to assist in therapy planning for future patients.

### BACKGROUND

1. Field of the Invention

The present invention relates to in vitro diagnostic devices and methods. More particularly, the invention relates to in vitro diagnostic devices and methods for assessing effects of treatments on channelopathies such as cardiac channelopathies.

2. Description of Related Art

Channelopathies are diseases caused by mutations of ion channel genes, ion channel-associated genes, or a disturbed function of ion channels. Cardiac channelopathies are a particularly insidious form of these types of diseases. Cardiac channelopathies may cause sudden cardiac death and include diseases like long-QT-syndrome (LQTS), Short-QT-syndrome (SQTS), Brugada syndrome (BrS), and catecholaminergic polymorphic ventricular tachycardia (CPVT). A common form of cardiac channelopathy is LQTS, in which what is known as the QT phase of the heartbeat is extended. Carriers of LQTS may be more susceptible to arrhythmias and sudden cardiac death. Accordingly, an increased mortality rate is associated with the mutation. There are more than 160 QT-extending drugs, which are considered as risk drugs for LQT syndrome patients.

The constantly increasing number of uncovered genetic variations with different degrees of penetration along with additional risk factors such as exascerbating adverse drug reactions, side effects, or comorbidities compared to general population can impede the diagnosis, risk stratification, and therapy of the diseases. A large problem lies in the fact that the many channelopathies cannot be taken into consideration in drug development as hardly any new important drugs would be introduced onto the market for safety reasons (due to possible adverse reactions for patients with channelopathies). On the other hand, it is desired to take into consideration all population groups adequately including channelopathy carriers.

The need for further information on cardiac channelopathies is high as 1 person in 2000 may suffer from cardiac channelopathies and there are many different underlying mutations, which are not and cannot all be considered during routine development of drugs. The high safety standards required during routine drug development may hinder the marketability of new and important drugs. To help protect this specific cardiac channelopathy population, sensitive and specific diagnostic and risk stratification is needed.

Genetic testing alone, however, is not a solution as an individual's mutations are often unique and the same mutation can cause different effects based on the individual's genetic background. The genetic testing of channelopathies may be scientifically useful but has limited application to life-sustaining therapy strategies because genetic testing is of a probabilistic nature and not mechanistic or based on direct correlations. The publication “HRS/EHRA expert consensus statement on the state of genetic testing for the channelopathies and cardiomyopathies” (Ackermann et al., Europace 2011 August; 13(8) 1077-1090, which is incorporated by reference as if fully set forth herein) states that a genetic examination provides no sufficient evidence for the diagnosis, forecast and therapy of the channelopathy disease.

Thus, there is a need for more predictive diagnosis and individual compatibility tests in vitro. Particularly, there is a need for in vitro testing of therapies and drugs that are already in use. Results of such testing may provide information useful to make better therapy decisions.

### SUMMARY

In certain embodiments, a compact in vitro diagnostic (IVD) device is used to provide in vitro model-based diagnosis and a basis for model-based therapy planning. The IVD device and an associated procedure may be used to assess the effect of a drug or a drug combination on a mutated ion channel or on action potentials of isogenic cardiomyocytes of a patient, or any other patient derived cell. In certain embodiment, the effects are assessed in a clinical environment. In certain embodiments, the assessed information for the patient (e.g., pharmaco-genomic knowledge obtained for the patient) is provided to a database of assessment results. The database may collect the assessment results and other patient data (e.g., medical history, genetic background, etc.) for a plurality of patients and be used to assist in future therapy decisions (e.g., model-based therapy planning).

In certain embodiments, the IVD device is based on a patch clamp instrument. For example, the IVD device may be based on a CytoPatch™ (Cytocentrics, Inc.) patch clamp instrument. In certain embodiments, the IVD device is used to diagnose the effect a drug or a drug combination may have on a patient by using a patch clamp diagnostic procedure to assess in vitro effects of the drug or drug combination on electrophysiological phenotype of cardiomyocytes (CMs) derived from patient-specific induced pluripotent stem cells (ps-iPSCs). In some embodiments, the IVD device and diagnostic procedure is used to assess a correlation between in vitro electrophysiological phenotype of CMs derived from ps-iPSCs and the patient's clinical symptoms and/or medical history.

### BRIEF DESCRIPTION OF THE DRAWINGS

Features and advantages of the methods and apparatus of the present invention will be more fully appreciated by reference to the following detailed description of presently preferred but nonetheless illustrative embodiments in accordance with the present invention when taken in conjunction with the accompanying drawings in which:

FIG. 1 depicts a flowchart for an embodiment of an in vitro diagnosis procedure.

FIG. 2 depicts a block diagram of one embodiment of an exemplary computer system.

FIG. 3 depicts a block diagram of one embodiment of a computer accessible storage medium.

While the invention is susceptible to various modifications and alternative forms, specific embodiments thereof are shown by way of example in the drawings and will herein be described in detail. The drawings may not be to scale. It should be understood that the drawings and detailed description thereto are not intended to limit the invention to the particular form disclosed, but to the contrary, the intention is to cover all modifications, equivalents and alternatives falling within the spirit and scope of the present invention as defined by the appended claims.

The headings used herein are for organizational purposes only and are not meant to be used to limit the scope of the description. As used throughout this application, the word “may” is used in a permissive sense (i.e., meaning having the potential to), rather than the mandatory sense (i.e., meaning must). The words “include,” “including,” and “includes” indicate open-ended relationships and therefore mean including, but not limited to. Similarly, the words “have,” “having,” and “has” also indicated open-ended relationships, and thus mean having, but not limited to. The terms “first,” “second,” “third,” and so forth as used herein are used as labels for nouns that they precede, and do not imply any type of ordering (e.g., spatial, temporal, logical, etc.) unless such an ordering is otherwise explicitly indicated. For example, a “third die electrically connected to the module substrate” does not preclude scenarios in which a “fourth die electrically connected to the module substrate” is connected prior to the third die, unless otherwise specified. Similarly, a “second” feature does not require that a “first” feature be implemented prior to the “second” feature, unless otherwise specified.

Various components may be described as “configured to” perform a task or tasks. In such contexts, “configured to” is a broad recitation generally meaning “having structure that” performs the task or tasks during operation. As such, the component can be configured to perform the task even when the component is not currently performing that task (e.g., a set of electrical conductors may be configured to electrically connect a module to another module, even when the two modules are not connected). In some contexts, “configured to” may be a broad recitation of structure generally meaning “having circuitry that” performs the task or tasks during operation. As such, the component can be configured to perform the task even when the component is not currently on. In general, the circuitry that forms the structure corresponding to “configured to” may include hardware circuits.

Various components may be described as performing a task or tasks, for convenience in the description. Such descriptions should be interpreted as including the phrase “configured to.” Reciting a component that is configured to perform one or more tasks is expressly intended not to invoke 35 U.S.C. §112 paragraph (f), interpretation for that component.

The scope of the present disclosure includes any feature or combination of features disclosed herein (either explicitly or implicitly), or any generalization thereof, whether or not it mitigates any or all of the problems addressed herein. Accordingly, new claims may be formulated during prosecution of this application (or an application claiming priority thereto) to any such combination of features. In particular, with reference to the appended claims, features from dependent claims may be combined with those of the independent claims and features from respective independent claims may be combined in any appropriate manner and not merely in the specific combinations enumerated in the appended claims.

### DETAILED DESCRIPTION OF EMBODIMENTS

A model-based in vitro diagnosis of the effect of a drug or drug combination may be used prior to the need for medication. The patient may have a need for the medication that is vital (e.g., life saving) but providing medication based solely on probabilistic assumptions (e.g., genetic testing models) may be life-threatening due to unknown pre-existing conditions or symptoms. The model-based in vitro diagnosis may be used to provide medication in an improved manner by providing a mechanistic approach for assessment of medication effects on a patient prior to providing the medication to the patient. Thus, using the model-based in vitro diagnosis may reduce the potential for severe side-effects and/or deaths. In some embodiments, the effects of multiple medications and/or combinations of medications are assessed using the model-based in vitro diagnosis prior to providing the medication.

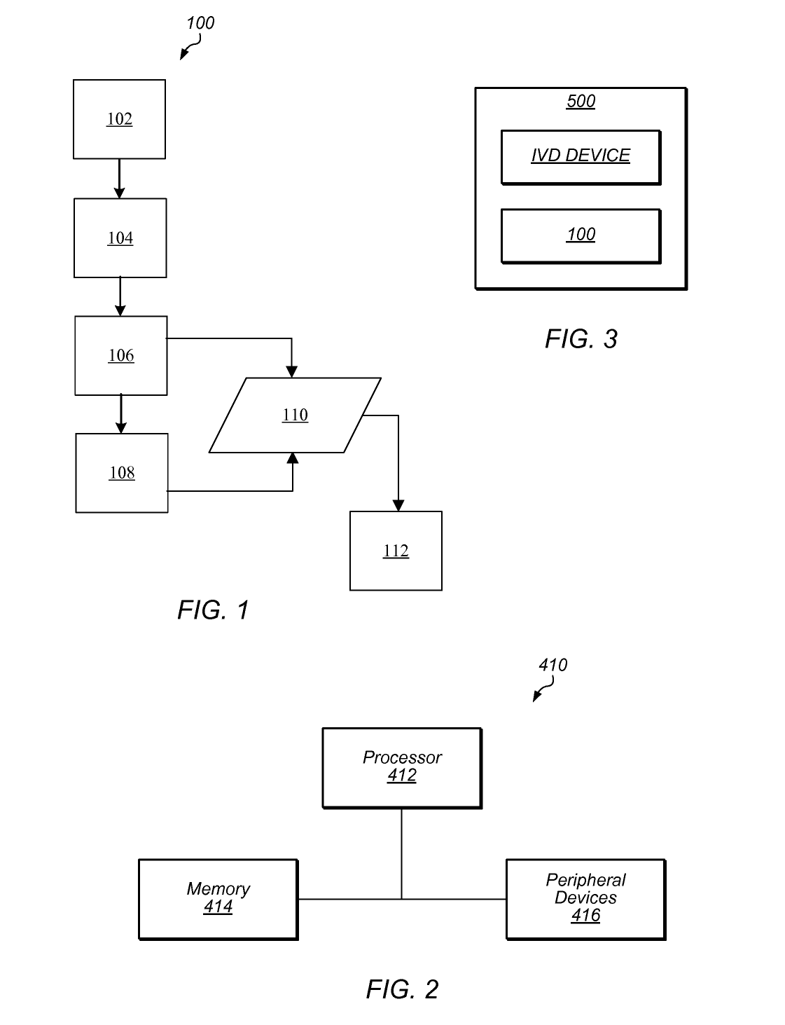

FIG. 1 depicts a flowchart for an embodiment of in vitro diagnosis procedure 100. In certain embodiments, in vitro diagnosis procedure 100 is used for treatment diagnosis for patients with cardiac channelopathies. Procedure 100 may be used following a differential diagnosis of a patient. The differential diagnosis may indicate that the patient likely has a cardiac channelopathy. In certain embodiments, after the diagnosis, cardiomyocytes (CMs) may be derived from patient-specific induced pluripotent stem cells (ps-iPSCs) in 102. An example of a method for deriving cardiomyocytes from ps-iPSCs is disclosed in “Patient-specific induced pluripotent stem-cell models for long-QT syndrome”: Moretti et al. New England Journal of Medicine 2010 Oct. 7; 363(15) 1397-1409, which is incorporated by reference as if fully set forth herein.

The ps-iPSCs may be obtained directly from a patient. In certain embodiments, the ps-iPSCs are obtained from the patient in a clinical setting (e.g., a medical clinic, hospital, or clinical laboratory). Because the cardiomyocytes are derived from patient-specific iPSCs, the cardiomyocytes are also patient-specific and may be called patient-specific iPSC cardiomyocytes (ps-iPSC-CMs). In some embodiments, the cardiomyocytes are derived from the transient transfection of the mutated gene in non-patient specific cell line (e.g., stem cell lines transiently transfected with patient DNA). In some embodiments, only one or several relevant genes of the patient are stably or transiently expressed in an expression cell line for the assessment of the electrophysiological phenotype of these patient-specific genes.

After derivation of the cardiomyocytes, the electrophysiological phenotype of the cardiomyocytes may be assessed in vitro in 104. In certain embodiments, an in vitro diagnostic (IVD) device is used to assess the electrophysiological phenotype of the cardiomyocytes. The assessment of the electrophysiological phenotype may include measurement of transmembrane potential and/or transmembrane currents. For example, the IVD device may be a transmembrane potential assay device to assess the electrophysiological phenotype of the cardiomyocytes.

In certain embodiments, the IVD device (e.g., the transmembrane potential assay device) is a patch clamp device. In some embodiments, the IVD device is an automated patch clamp device based on, for example, the CytoPatch™ patch clamp instrument (e.g., a modified-version of the CytoPatch™ patch clamp instrument). The CytoPatch™ patch clamp instrument and Cytocentering™ technique (method) is disclosed in U.S. Pat. No. 7,361,500 to Stett et al., which is incorporated by reference as if fully set forth herein.

The CytoPatch™ patch clamp instrument and Cytocentering™ technique allow individual cells from a suspension to be selected according to at least one criterion (e.g., a cell parameter). The selected individual cells may be positioned and immobilized from the suspension at a measurement site (e.g., at the opening). The immobilization may be carried out either via a suction channel (e.g., using hydrodynamic low pressure in the suction channel) and/or via a functional coating. The immobilized cells may be electrically contacted using an electrode. To contact the immobilized cells, a hydrodynamic low pressure is generated to act on a cell membrane through a contact tip projected into the opening. The hydrodynamic low pressure in the suction channel and the hydrodynamic low pressure acting on the cell membrane (e.g., the contact channel) may be independently controlled. Because the pressure in the suction channel and the contact channel are controlled independently, the positioning (and immobilization) and/or contacting of the cells may be automated and multiple cells (e.g., a plurality of cells) may be automatically assessed in vitro using the CytoPatch™ patch clamp instrument and Cytocentering™ technique . In some embodiments, immobilizing and contacting a plurality of cells in the CytoPatch™ patch clamp instrument includes independently controlling hydrodynamic low pressures on a plurality of patch pipettes, with each patch pipette surrounded by a suction pipette, to automatically assess the plurality of cells in vitro using the CytoPatch™ patch clamp instrument.

In certain embodiments, the IVD device is a CytoPatch™ patch clamp instrument modified to be user-friendly (e.g., suitable for use by clinical personnel in a clinical environment). In 104, the IVD device may perform a patch clamp technique (e.g., patch clamp method) for assessing the electrophysiological phenotype of the cardiomyocytes. In certain embodiments, the IVD device performs an embodiment of the Cytocentering™ technique (Cytocentrics, Inc.), described above, to assess the electrophysiological phenotype of the cardiomyocytes.

Following assessment of the electrophysiological phenotype of the cardiomyocytes, the IVD device may be used to assess a correlation or effect of a drug or a drug combination on the electrophysiological phenotype of the cardiomyocytes in 106. The drug or drug combination may include FDA approved(e.g., licensed or authorized) and/or yet to be approved (non-authorized, experimental, or repositioned) drugs or FDA approved drugs licensed for other indications (uses) than the particular channelopathy or clinical syndromes of the current patient (e.g., off-licensed use). The drugs or drug combinations to be tested in the IVD device may be determined based on other factors (e.g., other diagnoses or general treatment plans intended to help the patient with cardiac channelopathy-induced disease). In some embodiments, the drugs or drug combinations to be tested may include newly developed drugs (e.g., drugs in an experimental testing phase).

Using the IVD device allows the effect of the drug or drug combination to be assessed in vitro. For example, the pharmacology of the electrophysiological phenotype of the cardiomyocytes or the pharmacological modulation of the electrophysiological phenotype of the cardiomyocytes is assessed in vitro using the IVD device. In certain embodiments, the IVD device assesses the effect of the drug or drug combination on ion channels in the cardiomyocytes. For example, the IVD device may assess the effect of the drug or drug combination in cardiac ion channel currents such as, but not limited to, Ica, INav peak and late, Ikr, Iks, Ito, and Ikl.

In certain embodiments, the IVD device uses electrophysiological variables to assess patient-specific and mutation-specific effect of the drug or drug combinations. Electrophysiological variables may include, but not be limited to, amplitude, tail activation, voltage dependency, tail deactivation, real time IV (in vitro) plot/dynamic ramp, dose-response curves, and current clamp at action potentials. In certain embodiments, the mutation-specific changes for cardiac channelopathies include mutation-specific changes in ion channels that have been previously demonstrated. For example, the mutation-specific changes in HERG (IKr) ion channel and a cardiac potassium ion channel gene (KCNQ1) (IKs)) have been previously demonstrated

In some embodiments, the effect of the drug or drug combination is assessed using intracellular transmembrane potential measurement and/or intracellular transmembrane current measurement. In some embodiments, the effect of the drug or drug combination is assessed using extracellular field potential measurement. In addition, other techniques may be used to assess the effect of the drug or drug combination. Examples of techniques include, but are not limited to, MEA (micro-electrode array) technology, impedance measurement, optical measurement (e.g., fluorescence-optical measurement, voltage sensitive dye measurement, or optogenetically enabled measurement), and surface sensor methods.

Assessing the effect of the drug or drug combination on the electrophysiological phenotype of the cardiomyocytes may demonstrate the effect of the drug or drug combination in treating a specific patient's cardiac channelopathies. For example, the IVD device provides data or information that may be used to assess the interaction and effectiveness specific drugs or drug combinations have on treating the specific patient's cardiac channelopathies. The IVD device provides assessment of the interaction of the drugs or drug combinations prior to actual use of the drugs or drug combinations on the patient (or a patient with similar characteristics such as a family member). Thus, non-desired or adverse reactions (e.g., patient death) may be avoided because of the assessment provided by the IVD device. For example, drug selection and dose selection may be assessed for the patient with the IVD device before drugs are provided to the patient.

In certain embodiments, in 108, a correlation is assessed between the drug effect on the electrophysiological phenotype of the cardiomyocytes assessed in 106 and clinical data. Clinical data may include patient-specific data such as, but not limited to, patient medical history, patient symptoms, and patient genetic background. The assessed correlation in 108 may provide an evidence-based diagnosis and treatment assessment for an individual patient. The correlation assessed in 108 is associated with the patient's specific phenotype iso-genetic characteristics based on the in vitro pharmacology assessed in 106. In certain embodiments, the correlation assessment in 108 is performed using a computer processor. The computer processor may be included with the IVD device or coupled to the IVD device to receive information from the IVD device.

In certain embodiments, data from assessment 106 and/or assessment 108 is provided to database 110. Database 110 may store information from assessment 106 and/or assessment 108 for the specific patient. Thus, database 110 includes information about the effect of drugs or drug combinations on the electrophysiological phenotype of cardiomyocytes for the specific patient as well as clinical data about the specific patient. This information may be used to develop a therapy (treatment) plan for the specific patient or patients with similar characteristics.

In certain embodiments, procedure 100 is repeated for a plurality of patients with at least several patients having different characteristics (e.g., phenotype, genetics, channelopathies, etc.). Data from assessment 106 and/or assessment 108 for each patient may be provided to database 110. Thus, database 110 includes a plurality of data for different patients. The information in database 110 may be analyzed (assessed) to generate treatment and/or diagnosis algorithms for varieties of patients. The data in database 110 may also be analyzed to assess trends or other data algorithms useful in the treatment of cardiac channelopathies. For example, the in vitro electrophysiological phenotype (and its interaction with a drug or drug combination) may be analyzed together with clinical data for certain types of patients or all the patients in the database to provide a basis for risk stratification of the drugs or drug combinations tested in the IVD device.

In certain embodiments, a model-based therapy plan for a specific patient is determined based on the information in database 110. For example, the patient's in vitro electrophysiological phenotype may be assessed (e.g., using assessment 104 in FIG. 1) and used along with clinical data for the patient to model therapy (treatment) plan 112 for the patient based on the information in database 110. In some embodiments, therapy plan 112 is based on matching the patient to another (matched) patient with information in database 110. The matched patient may have similar electrophysiological phenotype and clinical data to the therapy plan patient. In some embodiments, therapy plan 112 is determined based on treatment and/or diagnosis algorithms generated from information in database 110.

In the case that therapy plan 112 includes the use of drugs or drug combinations in the treatment of the specific patient, procedure 100 may be used to assess (test) the effect of the drugs or drug combinations included in the therapy plan. For example, procedure 100 may be used to test how the drugs or drug combinations interact with the patient-specific cardiomyocytes and affect the electrophysiological phenotype of the cardiomyocytes. The model-based generation of therapy plan 112 provides a model-based in vitro diagnosis of the effect of a drug prior to the need for providing the drug (medication). Additionally, the model-based generation of therapy plan 112 improves the application of drug therapy by providing a patient-specific drug therapy instead of a generalized drug therapy, which can potentially be dangerous or even lethal depending on pre-existing conditions of the patient.

Procedure 100 and the assessment of the electrophysiological phenotype of the cardiomyocytes with the IVD device provide in vitro clinical diagnosis of cardiac channelopathies and potential treatments for clinical patients. Whereas genetic testing relies on inter-patient comparison of genetic data and ECG data, procedure 100 provides a mechanistic approach to an individual patient using the heart-beat like action potential recording in ps-iPSC-CMs. Thus, the outcome of therapeutic options (e.g., drug treatment) can not only be predicted, but also tested in vitro before being applied to the patient. Procedure 100 and the IVD device may reduce the patient's cardiac risk by improving therapy planning without testing different medicines on the patient himself under intensive care cardiac monitoring, which is the current standard treatment of high risk patients. Procedure 100 and the IVD device provide an evidence-based therapy decision for the clinical professional. Additionally, health insurance systems may incur less cost by less in-patient monitoring and fewer adverse drug reactions.

As procedure 100 is used over time and database 110 increases in patient information, the growing database may increase the ease-of-use and increase the predictive value of therapy plan 112. In some cases, database 110 may, when established, assist the pharmaceutical field in their in-silico-modelling approaches and decrease future drug development costs.

In addition, procedure 100 and the IVD device may reduce the use of defibrillators to treat patients suffering from cardiac channelopathy-induced disease. Most current patients suffering from cardiac channelopathy-induced disease receive a defibrillator that is implanted and has to be changed every 7 years. The initial implantation and changing of the defibrillator involves surgery, which increases the risk for complications and adverse effects. Procedure 100 and the IVD device may reduce the need for defibrillators through improved diagnosis and better treatment planning.

Further, the use of the CytoPatch™ technology in the IVD device automates what can be a very time consuming manual procedure. The manual procedure is performed by an electrophysiologist that is specifically trained over months in manual patch clamping (MPC). Trained technicians may operate the IVD device and free electrophysiologists from time-consuming and highly-frustrating manual research work to focus on experiment data analysis (e.g., analysis of database 110).

In some embodiments, the IVD device includes a cardiac action potential simulator. The cardiac action potential simulator may be used to assess patient-specific missings or alterings in certain ion channel conductivities. In some embodiments, the IVD device may be able to suggest compounds or medicines that can be tested for restoring the altered currents. In some embodiments, the IVD device uses current feedback algorithms (e.g., dynamic clamp) to restore the altered currents in vitro. Using the current feedback algorithms may provide additional evidence on the missing ion channel conductivities and/or provide a mathematical fit of the missing ion channel conductivities.

In some embodiments the IVD device uses use single ion channel recording techniques. In some embodiments, the IVD device uses capacity measurements to assess cellular membrane parameters. In some embodiments, the IVD assesses the contraction of the cardiomyocytes optically along with the patch clamp. In some embodiments, the IVD device assesses the electrophysiological phenotypic function of the cells (cardiomyocytes) by changing intracellular or extracellular buffers during the recordings. In some embodiments, the IVD device assesses the osmotic tolerance of the cells. For example, the IVD device may assess shrinking or expansion of the cells induced by osmotic changes in the buffer as assessed optically or by capacity measurement.

In some embodiments, the IVD device may include a cluster-strategy (e.g., clustering of data from different IVD devices). The cluster-strategy may allow electrophysiologists to remotely analyze data even from multiple locations. The IVD device may provide networked collaboration among multiple disciplines (e.g., physicians, cardiologists, oncologists, etc.).

Procedure 100 and the IVD device may be applied to cardiac channelopathies in areas ranging from personalized diagnostic practices of hospitals, to cardiac death risk stratification of athletes, and to better SID prevention. The risk a professional athlete may suffer from a channelopathy and thus, e.g., LQT during “work” is relatively high (1 out of 200). Newborn's cardiomyocytes may be tested from umbilical cord blood pre- or post-natal to assess a risk for SIDS due to cardiac channelopathies.

In certain embodiments, one or more process (procedure) steps described herein may be performed by one or more processors (e.g., a computer processor) executing instructions stored on a non-transitory computer-readable medium. For example, the IVD device or procedure 100, shown in FIG. 1, may have one or more steps performed by one or more processors executing instructions stored as program instructions in a computer readable storage medium (e.g., a non-transitory computer readable storage medium).

FIG. 2 depicts a block diagram of one embodiment of exemplary computer system 410. Exemplary computer system 410 may be used to implement one or more embodiments described herein. In some embodiments, computer system 410 is operable by a user to implement one or more embodiments described herein such as procedure 100, shown in FIG. 1. In the embodiment of FIG. 2, computer system 410 includes processor 412, memory 414, and various peripheral devices 416. Processor 412 is coupled to memory 414 and peripheral devices 416. Processor 412 is configured to execute instructions, including the instructions for procedure 100, which may be in software. In various embodiments, processor 412 may implement any desired instruction set (e.g. Intel Architecture-32 (IA-32, also known as x86), IA-32 with 64 bit extensions, x86-64, PowerPC, Sparc, MIPS, ARM, IA-64, etc.). In some embodiments, computer system 410 may include more than one processor. Moreover, processor 412 may include one or more processors or one or more processor cores.

Processor 412 may be coupled to memory 414 and peripheral devices 416 in any desired fashion. For example, in some embodiments, processor 412 may be coupled to memory 414 and/or peripheral devices 416 via various interconnect. Alternatively or in addition, one or more bridge chips may be used to coupled processor 412, memory 414, and peripheral devices 416.

Memory 414 may comprise any type of memory system. For example, memory 414 may comprise DRAM, and more particularly double data rate (DDR) SDRAM, RDRAM, etc. A memory controller may be included to interface to memory 414, and/or processor 412 may include a memory controller. Memory 414 may store the instructions to be executed by processor 412 during use, data to be operated upon by the processor during use, etc.

Peripheral devices 416 may represent any sort of hardware devices that may be included in computer system 410 or coupled thereto (e.g., the IVD device, storage devices, optionally including computer accessible storage medium 500, shown in FIG. 3, other input/output (I/O) devices such as video hardware, audio hardware, user interface devices, networking hardware, etc.).

Turning now to FIG. 3, a block diagram of one embodiment of computer accessible storage medium 500 including one or more data structures representative of data or input associated with the IVD device and one or more code sequences representative of procedure 100 is shown. Each code sequence may include one or more instructions, which when executed by a processor in a computer, implement the operations described for the corresponding code sequence. Generally speaking, a computer accessible storage medium may include any storage media accessible by a computer during use to provide instructions and/or data to the computer. For example, a computer accessible storage medium may include non-transitory storage media such as magnetic or optical media, e.g., disk (fixed or removable), tape, CD-ROM, DVD-ROM, CD-R, CD-RW, DVD-R, DVD-RW, or Blu-Ray. Storage media may further include volatile or non-volatile memory media such as RAM (e.g. synchronous dynamic RAM (SDRAM), Rambus DRAM (RDRAM), static RAM (SRAM), etc.), ROM, or Flash memory. The storage media may be physically included within the computer to which the storage media provides instructions/data. Alternatively, the storage media may be connected to the computer. For example, the storage media may be connected to the computer over a network or wireless link, such as network attached storage. The storage media may be connected through a peripheral interface such as the Universal Serial Bus (USB). Generally, computer accessible storage medium 500 may store data in a non-transitory manner, where non-transitory in this context may refer to not transmitting the instructions/data on a signal. For example, non-transitory storage may be volatile (and may lose the stored instructions/data in response to a power down) or non-volatile.

It is to be understood the invention is not limited to particular systems described which may, of course, vary. It is also to be understood that the terminology used herein is for the purpose of describing particular embodiments only, and is not intended to be limiting. As used in this specification, the singular forms “a”, “an” and “the” include plural referents unless the content clearly indicates otherwise. Thus, for example, reference to “a device” includes a combination of two or more devices and reference to “a drug” includes mixtures of drugs.

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. A method for in vitro diagnosis of a channelopathy, comprising:
assessing, in vitro using a transmembrane potential or current assay device, an effect of one or more drugs on one or more cells derived from a patient, wherein the drugs are contemplated to be potentially used in a medical treatment of the patient; and
providing patient-specific information based on the assessed effect.

2. The method of claim 1, wherein the transmembrane potential or current assay device comprises a patch clamp device.

3. The method of claim 2, further comprising immobilizing and contacting a plurality of cells in the patch clamp device using independently controlled hydrodynamic low pressures such that the plurality of cells are assessed automatically in vitro using the patch clamp device.

4. The method of claim 1, wherein the patient-specific information can be used for planning a medical treatment plan for the patient.

5. The method of claim 1, wherein the cells derived from the patient comprise cardiomyocytes derived from patient-specific induced pluripotent stem cells.

6. The method of claim 1, wherein the assessed effect comprises an assessed effect of an electrophysiological phenotype of any of the cells derived from the patient.

7. The method of claim 1, wherein the cells derived from the patient comprise a genetic mutation at cardiac ion channels.

8. The method of claim 1, wherein at least one of the drugs comprises an FDA approved drug.

9. The method of claim 1, wherein at least one of the drugs comprises an experimental drug.

10. The method of claim 1, wherein the effect is assessed using an intracellular transmembrane potential measurement.

11. The method of claim 1, wherein the effect is assessed using an intracellular transmembrane current measurement.

12. A method for in vitro diagnosis , comprising:
assessing, in vitro using a patch clamp device, an effect of one or more drugs on one or more cells derived from a patient, wherein the drugs are contemplated to be potentially used in a medical treatment of the patient;
assessing a correlation between the assessed effect and clinical data for the patient; and
generating one or more potential treatment plans for the patient based on the correlation.

13. The method of claim 12, wherein the cells derived from the patient comprise cardiomyocytes derived from patient-specific induced pluripotent stem cells.

14. The method of claim 12, wherein the assessed effect comprises an assessed effect of an electrophysiological phenotype of any of the cells derived from the patient.

15. The method of claim 12, wherein the cells derived from the patient comprise a genetic mutation at cardiac ion channels.

16. The method of claim 12, wherein the clinical data comprises medical history and genetic background of the patient.

17. The method of claim 12, wherein at least one of the treatments comprises a medication treatment for the patient.

18. The method of claim 12, further comprising immobilizing and contacting a plurality of cells in the patch clamp device using independently controlled hydrodynamic low pressures such that the plurality of cells are assessed automatically in vitro using the patch clamp device.

19. A method for generating a potential treatment for a patient with a channelopathy, comprising:
assessing, in vitro using a patch clamp device, an electrophysiological phenotype of one or more cells derived from a patient;
accessing data from a database, wherein the data comprises clinical data and drug interaction data for a plurality of patients, the drug interaction data comprising in vitro assessed data of an effect of one or more drugs on one or more cells derived from the plurality of patients; and
generating a potential treatment for the patient based on an analysis of the assessed electrophysiological phenotype and the accessed data.

20. The method of claim 19, wherein the in vitro assessed data of the effect of one or more drugs on one or more cells derived from the plurality of patients is obtained using one or more patch clamp devices.

21. The method of claim 19, wherein generating the potential treatment for the patient comprises accessing data from the database for a selected patient from the plurality of patients having similar electrophysiological phenotype and clinical data to the patient.

22. The method of claim 19, wherein generating the potential treatment for the patient comprises determining the potential treatment based on treatment and/or diagnosis algorithms generated from the data in the database.

23. The method of claim 19, wherein the patch clamp device comprises an automated patch clamp device.

24. A method for in vitro diagnosis, comprising:
assessing, in vitro, an effect of one or more drugs on one or more cells derived from a patient; and
providing patient-specific information based on the assessed effect.
